(19) United States
(12) Patent Application Publication
(10) Pub. No.: US 2014/0005463 A1
(43) Pub. Date: Jan. 2, 2014

(54) ASSEMBLY AND METHOD FOR CONFORMAL PARTICLE RADIATION THERAPY OF A MOVING TARGET

(71) Applicant: Yves Jongen, Louvain-la-Neuve (BE)

(72) Inventor: Yves Jongen, Louvain-la-Neuve (BE)

(73) Assignee: Ion Beam Applications, Louvain-Ia-Neuve (BE)

(21) Appl. No.: 13/552,405

(22) Filed: Jul. 18, 2012

Related U.S. Application Data
(60) Provisional application No. 61/665,419, filed on Jun. 28, 2012.

(30) Foreign Application Priority Data
Jun. 28, 2012 (EP) EP12174160.7

(51) Int. Cl. A61N 5/10

(52) U.S. Cl. CPC A61N 5/107; A61N 5/1037; A61N 5/1039; A61N 5/1077; A61N 5/1043
USPC 600/1

(57) ABSTRACT

Methods and assembly for delivering a particle beam dose to a moving target are provided. The assembly comprises breath holding means for holding a patients breath during the delivery of the particle beam dose. With the assembly and method of invention, visualization means are provided to the patient to visualize the moving target such that the patient can actively contribute to the target positioning before actuating the breath holding means and starting the delivery of the particle beam.

### BACKGROUND OF THE INVENTION

1. Field of the Invention

The invention relates generally to particle radiation therapy. More particularly, the present invention relates to an assembly and method for irradiating a moving target in a patient with a particle beam.

2. Description of the Related Art

Several types of particle therapy assemblies for irradiating targets with a particle beam are known. An example of a particle beam assembly is using a scanning device which comprises scanning magnets for directing and delivering the beam to an area of the target. By directing and delivering the beam sequentially to multiple scanning positions an entire target can be irradiated with the particle beam. The number of positions to be irradiated to cover the entire target depends on the target size and the particle beam size. In general, the scanning positions are defined as (x,y) coordinates in an X-Y scanning plane, which is a plane perpendicular to a main beam direction Z, said main beam direction Z being a direction followed by the particle beam at an exit of the scanning device when the scanning magnets are not energized.

Intra-fractional target motions, which are mainly caused by respiration of the patient, require adaptations in the particle therapy assembly in order to satisfy requirements related to dose conformation. Indeed, not taking into account target motion results in a blurring of the dose gradients from target volume to normal (healthy) tissue and, in addition, during target motion the radiological beam path length can change, which could result in under-dosage or over-dosage of the target volume. The known different types of particle therapy assemblies adapted for irradiating moving targets with scanning beams are further discussed.

A first type of particle therapy assembly for irradiating moving targets is using the so-called beam gating technique. With this technique, based on a signal from a motion-monitoring device, the beam delivery to the target is controlled such that beam delivery only occurs during specific phases of the breathing cycle. Those assemblies are complex as synchronization is needed between the beam on/off controls and the phases of the breathing cycle. As the beam delivery only takes place during a fraction of the breathing cycle, the irradiation time is also increased.

A second type of particle therapy assembly for irradiating moving targets with a scanning device is using the so-called re-scanning or re-painting technique. With this technique the target is irradiated multiple times with a partial dose in order to smear out the effect of target motion. A disadvantage of such a technique is that healthy tissue may nevertheless be incidentally irradiated with partial doses. Another disadvantage is that the re-paintings are time consuming so that the overall treatment time is increased. A repainting technique is for example described in EP2392383.

A third type of particle assembly for irradiating moving targets is using the beam tracking technique. With this technique, the lateral position of the beam and/or beam energy are adjusted during beam delivery for compensating for target motion. This type of assembly is also complex and requires for a 4D treatment planning system. For example, in US2011105821, a system is provided to regulate the energy of the beam during the patient's irradiation depending on the detection of a target motion.

Hence there is a need for an improved assembly and method for irradiating moving targets with a particle beam. There is a need for an improved assembly and method for more time-efficient irradiation of tissue while minimizing incidental irradiation of healthy tissue.

### BRIEF SUMMARY OF THE INVENTION

An improved particle therapy assembly specifically adapted for irradiating moving targets is described. A method for irradiating a moving target in a patient with a particle beam is also described. One particularly significant aspect of the invention provides an assembly and method to overcome the drawbacks associated with the prior art, including but not limited to those discussed above. In particular, the assembly and method of the current invention allows for the precise irradiation of a moving target in a time-efficient and precise manner. In one embodiment, the present invention allows for the rapid identification and irradiation of a target; in some instances within the time period of a single breath of a patient.

These and other aspects of the invention are achieved with the assembly and methods as claimed.

The present invention provides an assembly and method comprising an imaging device for acquiring an actual position of the moving target while the patient is positioned in the irradiation treatment position. The imaging device has a display and displaying controls configured for displaying, in real time, information indicative of the actual position of the moving target and information indicative of a prescribed target position. The particle therapy assembly according to the invention also includes a breath holding means.

The patient can actively contribute to positioning the target by managing his breath based on the actual target position and prescribed target position displayed on the screen. When there is a match between the actual target position and the prescribed target position within a tolerance, the breath holding means are activated. The irradiation of the target is started in synchrony with the actuation of the breath holding means. In particular, an assembly is provided for conformal particle radiation therapy whereby the irradiation of the target is performed in a timeframe corresponding to a single breath hold period.

### BRIEF DESCRIPTION OF THE SEVERAL VIEWS OF THE DRAWINGS

These and further aspects of the invention will be explained in greater detail by way of example and with reference to the accompanying drawings in which:

FIG. 1 shows a schematic view of an example of particle therapy assembly;

FIG. 2 shows a schematic view of an example of a particle therapy assembly according to the invention;

FIG. 3 shows a schematic representation of an example of an energy filter;

FIG. 4 shows a schematic representation of multiple examples of filtering elements;

The figures are not drawn to scale. Generally, identical components are denoted by the same reference numerals in the figures.

### DETAILED DESCRIPTION OF THE INVENTION

The present invention will be more fully understood by reference to the Figures and the following description. The Figures and description below pertain to preferred embodiments of the present invention. Variations and modifications of these preferred embodiments and other embodiments within the scope of the invention can be substituted without departing from the principles of the invention, as will be evident to those skilled in the art.

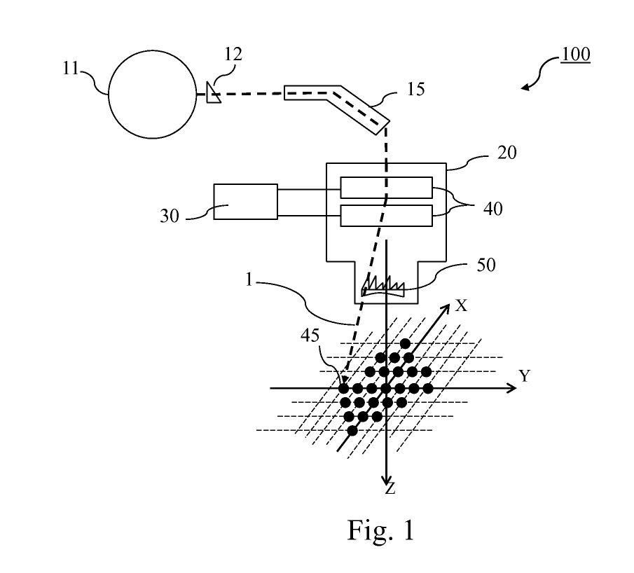

According to a first aspect of the invention a particle therapy assembly (100) is provided for irradiating moving targets. FIG. 1 shows a schematic view of an example of particle therapy assembly (100) according to the invention (not all components are shown on the figure). The assembly comprises a particle beam generator for generating a particle beam (e.g. proton or carbon ion beam). Such particle beam generator can be in a variety of forms. As one example, a particle beam generator includes a fixed energy cyclotron (11) and an energy degrader (12) for degrading the fixed energy such that a particle beam of a selected energy can be delivered. For example, beams with a proton generator can actually be delivered with an energy varying between 70 MeV and 230 MeV. For another example, the particle beam generator can be a synchrotron where the energy of the beam extracted from the synchrotron can be selected. The particle beam having a selected energy is then, using a transport system (15), further transported to a scanning device (20) (sometimes called nozzle). The scanning device (20) is adapted for delivering the particle beam in an appropriate form to a target volume within a patient. The scanning device functions to shape the dose distribution to the target volume to be irradiated. The scanning device (20) comprises one or more scanning magnets (40) for scanning the particle beam. The scanning device (20) will typically comprise two scanning magnets (40) for scanning the particle beam in an X-Y scanning plane, or, alternatively, a single double direction scanning magnet (40) can be used to scan the beam in the two directions X and Y.

In FIG. 1, a particle beam (1) is illustrated as a dashed line. The scanning device further comprises scanning control means (30) for controlling the scanning magnets (40). The scanning control means (30) are configured for scanning the particle beam by sequentially moving the particle beam to multiple scanning positions (45) situated in the X-Y scanning plane. This is illustrated in FIG. 1 where, as an example, a number of scanning positions (45) are shown in an X-Y plane. In general, the X-Y plane is perpendicular to a central axis Z of the nozzle (20), this central axis Z is defined as the direction the particles follow if the beam is not scanned (i.e. no current in the scanning magnets). In other words, the Z-axis corresponds to the central beam path. In general, the scanning plane X-Y is also defined as a plane going through the isocenter of the scanning device.

The scanning device (20) according to the invention may be mounted by a variety of methods known in the art. For example, the scanning device (20) can be mounted on a gantry for rotation about the isocenter. Alternative methods for mounting the scanning device (20) include installing the device (20) in a fixed beam line configuration or the scanning device (20) may be integrated in any other type of system configuration.

The particle therapy assembly (100) further comprises a patient support device (55), such as a couch, or any other device for positioning the patient in an irradiation treatment position. This irradiation treatment position is the position where the target is irradiated.

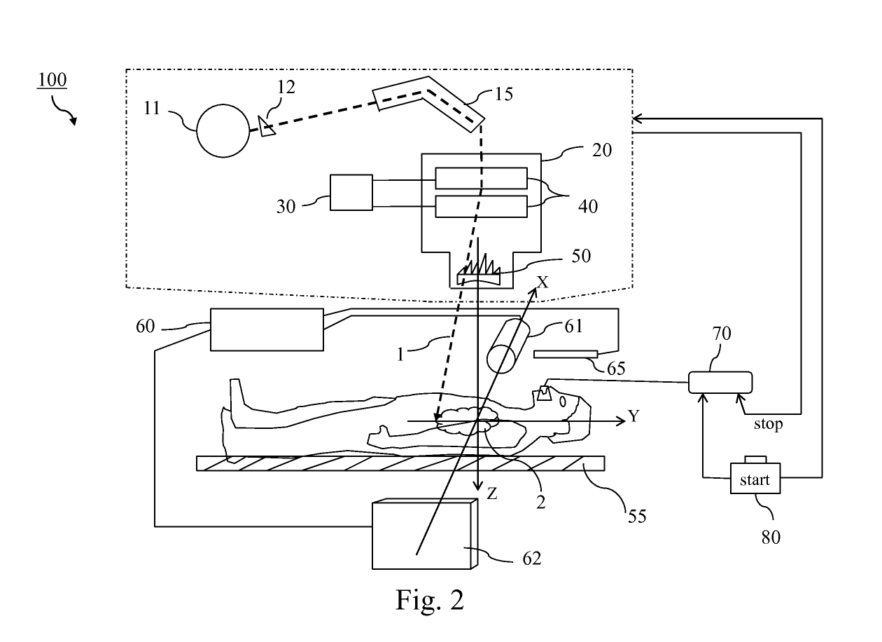

As shown schematically on FIG. 2, the particle therapy assembly according to the invention comprises means (60, 61, 62) for acquiring an actual position of the moving target while the patient is positioned in the treatment position. The imaging device further comprises a display (65) and displaying controls configured for displaying, in real time, information indicative of the actual target position and information indicative of a prescribed target position on said display (65).

The prescribed target position is defined by a medical doctor during the treatment planning phase.

The information indicative of the actual target position and the information indicative of the prescribed target position can, for example, be an outline of the contour of the target. Alternatively the information can, for example, be the centre point of the target. The imaging device may comprise, for example, a fluoroscopic imaging device. Such a fluoroscopic imaging device comprises an X-ray source (61) and an image receiver (62) that are, for example, located at 90° with respect to a central beam path through the nozzle (Z-axis shown in FIG. 2). Other direct imaging devices known in the art may be used.

Alternatively, instead of using a direct imaging method such as fluoroscopic imaging, an indirect method can be used for visualizing a moving target. For example an external optical tracking system can be used to track the external motion of the surface of the patient and those external motions can be correlated with the internal target motion.

The particle therapy assembly according to the invention further comprises breath holding means (70) for holding a breath of the patient while the patient is positioned in the irradiation treatment position. Typically, the breath holding means comprises an active breath control (ABC) device known in the art. With an ABC device, the patient breathes through a mouth-piece and a valve is used to temporarily block the airflow of the patient. The purpose is to have the breath of the patient blocked for a specified duration of time. Typically, that period of time will be about 20 seconds or less. During this period the irradiation of the target is performed using the scanning device (20) according to the invention.

The particle therapy assembly (100) comprises means for actuating (80) the breath holding means to start holding the patient's breath.

The particle therapy assembly further comprises means for starting the irradiation of the target with the particle beam. This action of starting the irradiation of the target is performed in relationship with the actuation of the breath holding means. In particular, when the breath holding means are actuated, the irradiation of the target with the particle beam also has to be started. These two actions can either be done separately by first actuation of the breath holding means and then starting the irradiation in a second step, or both together in one step as described below. The time between the actuation of the breath holding means and the start of the irradiation should however be as short as possible in order to keep the breath holding period as short as possible. The start of the irradiation is preferably performed within less than one second after the actuation of the breath holding means. For this purpose, two actuating buttons can, for example, be installed next to each other: one for actuating the breath holding means and one for starting the irradiation immediately after actuating the breath holding means.

However, preferably, the means for actuating (80) the breath holding means is coupled to the means for starting the irradiation such that the actuation of the breath holding and the start of delivering the particle beam are performed in collaboration.

Therefore, preferably, the assembly according to the invention comprises means for synchronizing the means for starting the irradiation and the means for actuating (80) the breath holding means. In this way, the start of the irradiation can be performed automatically without delay following the actuation of the breath holding means.

For example, the means for synchronizing comprise a common start button that triggers both the actuation of the breath-holding means and the start of the irradiation of the target. This common start button (80) is schematically represented in FIG. 2.

More preferably, the actuation of the breath holding means and the starting of the irradiation are performed simultaneously.

When the irradiation of the target is completed, the delivery of the particle beam is stopped and the breath-holding means are de-activated such that the patient can breathe normally. The synchronization means can, for example, also be configured for de-activating the breath holding means when the irradiation of the target is completed. This is illustrated schematically in FIG. 2 where the breath-holding means (70) receives a stop signal from the scanning control means (30) and/or controls of the particle beam generator when the irradiation of the target is completed.

In a preferred embodiment of the invention, the means for actuating breath holding means further comprises computing means (not shown) for other functions of the assembly described throughout the description. For example, the assembly preferably includes means for comparing the actual target position with the prescribed target position. The assembly also preferably includes means for providing a signal when the actual target position is located within a given tolerance of the prescribed target position.

This tolerance for positioning the target is for example 30 mm or 20 mm or 10 mm or 5 mm. The tolerance is in general smaller than 50 mm, preferably smaller than 30 mm and more preferably smaller than 20 mm.

For example, in operation the contours of the actual target position and the prescribed target position can be outlined on a display and when there is an overlap within the given tolerance between the two contours, a green signal is activated.

In a preferred embodiment, the imaging device comprises means for positioning the display (65) such that the patient can visually observe the display (65) while he is in the treatment position. The means for positioning the display (65) can, for example, be a movable telescopic arm connected to, for example, the ceiling or connected to the couch (55) or other patient support device, or connected to the scanning device (20). The display can, for example, be a flat panel type display. Alternatively, the means for positioning the display (65) can be a pair of glasses the patient wears and is configured such that the display is integrated in the pair of glasses.

In this way, having a display and the means for positioning the display such that the patient can see the screen, the patient can actively contribute to help to position the target in the right place (i.e. the irradiation treatment position) before delivering the particle beam to the target.

In addition to the display positioned to be viewed by the patient, a second display in the form of a screen can, for example, be provided to the radiotherapist or other person located in the treatment control room.

When the actual target position and the prescribed target position are in agreement within a tolerance, the breath holding means for holding the patients breath are actuated and the delivery of the particle beam is started. For performing the actuation, an actuation button can, for example, be used. As discussed before, this can be a single actuation button for both functions of actuating the breath holding means and starting the irradiation. This actuation button can also be a button to be selected on a computer screen.

In a first user scenario, the radio-therapist operates the actuation button and in second user scenario, the patient operates the actuation button.

In a preferred embodiment, the beam generator (11, 12) and the scanning device (20) are configured for irradiating the target with the particle beam in 20 seconds or less.

In a more preferred embodiment, the beam generator (11, 12) and the scanning device (20) are configured for irradiating the target with the particle beam in 10 seconds or less.

The particle therapy assembly (100) according to the invention may preferably include means (50) for modulating the energy of the particle beam as function of the scanning positions (45).

In modulating the energy of the particle beam, it is understood that the energy of a particle beam can be varied between a minimum and a maximum value such that the penetration depth of the beam in the target is varied. In general, the modulation of the energy of the particle beam, is performed either in steps or continuous between a minimum energy value and a maximum energy value. By varying the energy of the particle beam, the position of the Bragg peak in the target is varied. By adding several Bragg peaks having different positions in the target, i.e. by using multiple particle beams having different energies, a so-called Spread-Out-Bragg-Peak (SOBP) is generated in the target. To each of the Bragg peaks, contributing to the SOBP, a beam intensity weight is associated. As the modulation is performed as function of the scanning position, a different Spread-Out-Bragg-Peak profile can be generated in the target for a different scanning position. A different SOBP profile is generated by applying a different energy modulation, i.e. by providing different beam intensity weights for the various energies. Depending on the beam intensity weights chosen, the SOBP can be flat or it can have another non-flat shape. The minimum and maximum energy of the beam are determined based on the proximal and the distal range needed in the target for providing optimum dose conformation.

Because the energy modulation can be varied independently in the X and in the Y directions, such a means for modulating the energy permits to achieve high dose conformation to the target volume, regardless of the 3D shape of the target volume.

In a preferred embodiment according to the invention, the means (50) for modulating the energy of the particle beam are installed downstream of the scanning magnets (40). In this context, “downstream” is defined with respect to the beam direction, i.e. the beam is first travelling through the scanning magnets before reaching the means (50) for modulating the energy of the particle beam.

With the preferred particle therapy assembly according to the invention, due to the downstream energy modulation, the target volume can be irradiated by performing a single scan, i.e. the sequence of delivering the beam to the multiple scanning positions needs only to be performed one time for delivering a prescribed dose to the target. Hence, the irradiation period can be significantly reduced. With the preferred scanning device (20) according to the invention, the irradiation period for irradiating, for example, a one litre target volume is 20 seconds or less. Typical irradiation periods with current scanning devices using conventional scanning techniques are of the order of one to two minutes.

With those conventional scanning techniques the target is divided in layers (typically 10 to 20 layers) and the irradiation of the target is performed layer per layer.

Each layer corresponds to a particle energy and after each layer the energy of the beam needs to be varied upstream. The change of energy typically takes one second and this greatly increases the overall irradiation time period of the prior art techniques.

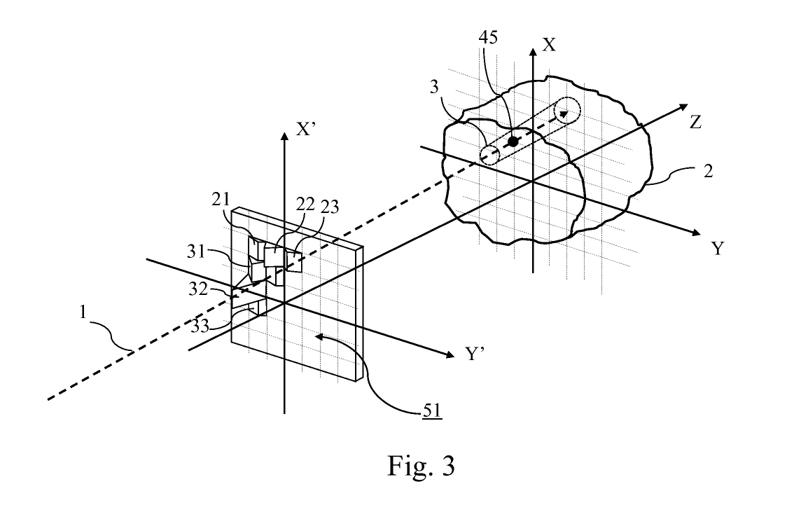

In a more preferred embodiment of the invention, the means (50) for modulating the energy comprises an energy filter (51) having a plurality of filtering elements. Each of the plurality of filtering elements is configured for modulating the energy of the particle beam and is associated to one of the scanning positions (45) of the particle beam in the X-Y plane. The energy filter (51) is further configured and located such that when the beam is directed to a scanning position (45), the associated filtering element is being crossed by the particle beam. This is illustrated in FIG. 3, where an example of such a filter configuration is shown. This exemplary filter (51) comprises a plurality of individual filtering elements (21, 22, 23, . . . , 31, 32, 33, . . . ) which are individually arranged in a transversal plane according to an X′,Y′ grid. For the sake of clarity, not all filtering elements are shown on this figure. In this example, both X′ and Y′ directions are perpendicular to each other and the filtering elements (21, 22, 23, . . . , 31, 32, 33, . . . ) are arranged according to an orthogonal grid (shown with dotted lines), but other arrangements may of course also be used, such as non-orthogonal grids for example.

Taking into account the direction of the scanned particle beam (1) (one scanned beam (1) is indicated by a dashed line on FIG. 3), the skilled person will understand that each filtering element, such as element (23) for example, will correspond to a particular region (3) in the target volume (2) for which a specific Spread-Out-Bragg-Peak profile is to be delivered. For a given filtering element (23), the corresponding region (3) in the target volume (2) is that part of the target volume (2) which will be irradiated with those particles having passed through the given filtering element (23).

Each of the filtering elements (21, 22, 23, 31, 32, 33) comprises multiple sub-filtering elements (not shown) having different material thicknesses. In this way, when the filtering element is crossed with the particle beam, a distribution of particle energies is provided at the output of the filtering element (21, 22, 23, 31, 32, 33,).

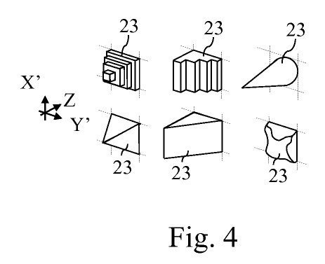

FIG. 4 shows various possible three-dimensional (“3D”) shapes for a filtering element (21, 22, 23, 31, 32, 33) of the energy filter (50) of FIG. 3. As shown on FIG. 4, a filtering element (23) may for example have the shape of a 3D pyramid, or of a 3D staircase, or of a 3D cone, each of which having either stepped or continuous lateral slopes. A filtering element (23) may also have a more complex 3D shape, such as the shape shown in the bottom right part of FIG. 4 for example.

For a given 3D shape of a filtering element, for example a 3D pyramid, the detailed geometrical dimensions (“the geometry”) of said filtering element, such as the number of steps as well as the height and the width (frontal surface) of each step, are determined in advance as a function of the desired SOBP (Spread-Out-Bragg peak) profile in the corresponding region (3) of the target volume (2). To this end, an analytical transfer function of a filtering element may be used and an optimization loop may make several iterations with this transfer function until obtaining the desired SOBP profile in the corresponding region of the target volume (2). Such methods are known from the skilled person for calculating the known ridge filters for example. Accordingly, to each region in the target volume (3) is associated a corresponding filtering element with a particular geometry. Having determined the geometries of all individual filtering elements (21, 22, 23, . . . , 31, 32, 33, . . . ), dedicated to a particular target volume (i.e. to a particular patient) can be built, for example by stereolithography.

Typically, the energy filter (51) has lateral dimensions (in the X′Y′ plane) which substantially correspond to the frontal surface of the target volume (2). For a target volume having maximum outer dimensions of 10 cm×10 cm×10 cm (according to X, Y, Z), the energy filter may for example have overall outer dimensions of about 10 cm×10 cm (according to X′, Y′).

The number of filtering elements arranged in the transversal plane as well as their respective dimensions may be freely chosen and will depend on the required accuracy of the dose conformity.

For performing the function of shifting the energy as function of the scanning position, the energy filter (51) comprises, for example, a so-called range compensator, which is well known to the person skilled in the art. A range compensator (sometimes called bolus) is a device specifically adapted according to the shape of the target such that the distal range of the beam is adjusted according to the shape of the target.

In a more preferred embodiment, the particle therapy assembly (100) according to the invention further comprises computing means for computing an expected irradiation period. How long a patient will be able to hold his breath will vary significantly from patient to patient. In general, it is known that a patient should be capable to hold his breath between 10 seconds and 20 seconds.

Therefore, it is important to know, before launching the particle beam irradiation, how much time the irradiation will take in order to be sure that the irradiation can be performed within a single breath holding period the particular patient is capable of performing. This information of expected irradiation period and/or an additional count-down of the remaining irradiation time can be displayed on the display, which is visible to the patient. It will comfort the patient during the irradiation to know the remaining time he has to hold his breath. The time period to perform the irradiation depends on a number of factors including the number of scan positions (45), the dose to be delivered to the target for each scanning position, the maximum beam current the particle beam generator (11, 12) can deliver, and the time to switch from scanning position to scanning position.

The dose to be delivered for each scan position is determined by a treatment planning system. This dose is either directly expressed in machine units or monitor units (MU) or the dose is provided in units of Gy (Gray). In the later case, a translation is typically first made into monitor units. The computing means will then define the beam current the particle generator should deliver. The computing means comprises a table defining the maximum beam current the particle beam generator can produce as function of the beam energy. The computing means, taking into account the maximum beam current, then calculates for each scanning position what the irradiation time is based on the dose, expressed in monitor units, to be delivered for that scanning position and a calibration factor defining the relation between monitor units and the particle beam intensity. This calibration factor will depend on the energy modulation and can hence vary from scanning position to scanning position. The calibration factor can be experimentally determined and stored in calibration tables. The computing means comprises these calibration tables defining this relation between monitor units and particle beam intensity. By summing the irradiation times of all scanning positions, and taking into account the time for switching the particle beam to all scanning positions, the expected irradiation time period is computed by the computing means.

The expected irradiation period is preferably displayed on the display (65).

According to a second aspect of the invention, a method is provided for irradiating a moving target in a patient with a particle beam using a particle therapy assembly (100). The method according to the invention for irradiating a moving target in a patient with a particle beam comprises the steps of
a. positioning said patient in an irradiation treatment position;
b. providing breath holding means (70) for holding the patient's breath;
c. providing an imaging device (60, 61, 62, 65) configured for acquiring an actual position of said moving target while the patient is in said irradiation treatment position, said imaging device comprising a display (65) and displaying controls configured for displaying, in real time, information indicative of the actual position of said moving target and information indicative of a prescribed target position on said display (65);
d. positioning said display (65) such that the patient can visually observe the display (65) while said patient is located in said irradiation treatment position;
e. displaying, in real time, the information indicative of the actual target position together with the information indicative of the prescribed target position on said display (65);
f. managing the patient's respiration such that the actual target position matches the prescribed target position within a tolerance, said managing being performed based on said information indicative of the actual target position and said information indicative of said prescribed target position;
g. actuating the breath holding means to start holding the patient's breath when said actual target position matches the prescribed target position within a tolerance;
h. starting the irradiation of the target with said particle beam when said actual target position matches the prescribed target position within said tolerance.

In a preferred method, the method includes
i. completing the irradiation of the target within a single breath holding period.

More particularly, the method is used with a particle therapy assembly (100) using a scanning device. Such a particle therapy assembly (10) comprises
a particle beam generator (11, 12) for generating a particle beam;
a scanning device (20) for scanning the target with the particle beam, said scanning device comprises
   one or more scanning magnets (40) for scanning the particle beam over an X-Y scanning plane;
   scanning control means (30) configured for scanning the particle beam by sequentially moving the particle beam to multiple scanning positions (45) situated in said X-Y scanning plane;

The method according to the invention using such a particle therapy assembly comprises the steps of
a. positioning said patient in an irradiation treatment position;
b. providing breath holding means (70) for holding the patient's breath;
c. providing an imaging device (60, 61, 62) for acquiring and visualizing an actual target position of said moving target while the patient is in said treatment position and said imaging device comprises a display (65) and displaying controls configured for displaying, in real time, information indicative of the actual target position and information indicative of a prescribed target position;
d. positioning said display (65) such that the patient can visually observe the display (65) while said patient is located in said treatment position;
e. displaying, in real time, the information indicative of the actual target position together with the information indicative of the prescribed target position on said display (65);
f. managing the patient's respiration such that the actual target position matches the prescribed target position within a tolerance, said managing being performed based on said information indicative of the actual target position and said information indicative of said prescribed target position;
g. actuating the breath holding means to start holding the patients breath when said actual target position matches the prescribed target position within a tolerance;
h. starting the irradiation of the target when said actual target position matches the prescribed target position within said tolerance.

In this way, by having the patient managing his respiration based on the information displayed on the display, the patient can actively vary the inflation level of his lungs to adjust or bring the actual position of the target close to the prescribed position. The prescribed position of the target is the position to irradiate with the particle beam as prescribed by the treatment planning. As an example, to help to compare the actual position and the prescribed position, the contours of the target in the actual position and the contours of the target in the prescribed position can be outlined on the display. As an example, a green signal indicating when the actual and prescribed positions are located within a given tolerance can be visualized on the display.

At that moment, when the actual position of the target corresponds, within some tolerance, to the prescribed position, the breath holding means is actuated and the delivery of the particle beam is started.

With the step of having the patient actively helping to have the actual target position matching the prescribed position, the reproducibility of the position of the target and the organs at risk can be greatly improved.

In a more preferred method according to the invention, the particle therapy assembly (100) further comprises means (50) for modulating an energy of the particle beam as function of the scanning positions (45) and the means (50) for modulating the energy are located downstream of all of said one or more scanning magnets (40).

As no upstream time consuming energy changes are needed, this allows a single scan for delivering a dose to the entire target volume to be performed. This greatly reduces the irradiation time period and hence allows the irradiation to be performed within a breath holding period. The breath-hold period is preferential equal or less than 20 seconds.

Hence, such a more preferred method using a particle therapy assembly (100) having means for modulating an energy which are located downstream of the scanning magnets, comprises advantageously completing the irradiation of the target within a single breath holding period.

Hence, there is no need for dividing the irradiation over multiple breath holding periods, thereby better ensuring patient compliance and minimizing the risk of irradiating healthy tissue.

In one user scenario, the actuation of the breath holding means is performed by the patient himself while being located in the treatment position. When the actual position corresponds within some tolerance to the prescribed position, the patient can push, for example, an actuation button to actuate the breath holding means.

In an alternative user scenario, the patient can adjust his breath using the visualization on the display of his moving target. The actuation of the breath holding means and start of irradiation is performed by another person, for example a radio-therapist located in the treatment control room who is observing the same visualization of the moving target.

Preferably, for both scenarios, the actuating of the breath holding and the starting the delivery of the particle beam are performed simultaneously. As mentioned before, as an example, a single actuation button can be used to simultaneously actuate the breath holding means and start the irradiation with the particle beam.

As another example, instead of using an actuation button, computer controls can perform a comparison between the actual position of the target and the prescribed position of the target. When both positions are located within a tolerance, the computer controls can then automatically actuate the breath holdings means and also trigger the start of the irradiation of the target.

The method disclosed above for treating moving targets with a particle therapy assembly is described for a scanning beam delivery device capable of performing the beam irradiation within a single breath-hold of 20 seconds or less.

More particularly, the method of invention is used with a particle therapy assembly (100) wherein the means (50) for modulating the energy comprises an energy filter (51) as described above.

The present invention has been described in terms of specific embodiments, which are illustrative of the invention and not to be construed as limiting. More generally, it will be appreciated by persons skilled in the art that the present invention is not limited by what has been particularly shown and/or described hereinabove. The invention resides in each and every novel characteristic feature and each and every combination of characteristic features.

Reference numerals in the claims do not limit their protective scope. Use of the verbs “to comprise”, “to include”, “to be composed of”, or any other variant, as well as their respective conjugations, does not exclude the presence of elements other than those stated. Use of the article “a”, “an” or “the” preceding an element does not exclude the presence of a plurality of such elements.

1. A particle therapy assembly for irradiating a moving target in a patient with a particle beam, said particle therapy assembly comprising
a particle beam generator for generating a particle beam;
a scanning device for scanning said target with said particle beam, said scanning device comprising
one or more scanning magnets for scanning the particle beam over an X-Y scanning plane;
scanning control means configured for scanning the particle beam by sequentially moving the particle beam to multiple scanning positions situated in said X-Y scanning plane;
a patient support device for positioning said patient in an irradiation treatment position;
an imaging device configured for acquiring an actual position of said moving target while the patient is in said irradiation treatment position, said imaging device comprising a display and displaying controls configured for displaying, in real time, information indicative of the actual position of said moving target and information indicative of a prescribed target position on said display;
breath holding means for holding a breath of said patient while said patient is positioned in said irradiation treatment position;
means for actuating said breath holding means to start holding the patient's breath;
means for starting an irradiation of the target with the particle beam.

2. A particle therapy assembly according to claim 1 wherein said particle therapy assembly further comprises means for positioning said display such that said patient can visually observe said information indicative of the actual position of said moving target and information indicative of a prescribed target position while said patient is in said irradiation treatment position.

3. A particle therapy assembly according to claim 1 wherein said display is in a display position which is such that said patient can visually observe said information indicative of the actual position of said moving target and said information indicative of a prescribed target position while said patient is in said irradiation treatment position.

4. A particle therapy assembly according to claim 1 further comprising
computing means for comparing in real time said actual target position with said prescribed target position, and;
means for providing a signal when said actual position matches said prescribed target position within a tolerance.

5. A particle therapy assembly according to claim 1 further comprising means for synchronizing said means for starting the irradiation and said means for actuating the breath holding means.

6. A particle therapy assembly according to claim 5 wherein said means for starting the irradiation and said means for actuating the breath holding means are configured such that said starting the irradiation is performed at the same time as said actuating the breath holding means.

7. A particle therapy assembly according to claim 1 wherein said imaging device comprises a fluoroscopic imaging device.

8. A particle therapy assembly according to claim 1 wherein said means for actuating the breath holding means comprises an actuation button operable by a human operator.

9. A particle therapy assembly according to claim 1 further comprising computing means for computing an expected irradiation time period for irradiation said target.

10. A particle therapy assembly according to claim 1 wherein said breath holding means comprises an active breath control (ABC) device.

11. A particle therapy assembly according to claim 1 further comprising means for modulating an energy of the particle beam as function of said scanning positions and wherein said means for modulating the energy are located downstream of all of said one or more scanning magnets.

12. A particle therapy assembly for irradiating a moving target of a patient with a particle beam, said assembly comprising
a particle beam generator for generating a particle beam;
a scanning device for scanning said target with said particle beam, said scanning device comprising
one or more scanning magnets for scanning the particle beam over an X-Y scanning plane;
scanning control means configured for scanning the particle beam by sequentially moving the beam to multiple scanning positions situated in said X-Y scanning plane;
an energy filter for modulating an energy of the particle beam as function of said scanning positions, wherein said energy filter is located downstream of said one or more scanning magnets, said energy filter having a plurality of filtering elements, each of said plurality of filtering elements being configured for modulating the energy of the particle beam and being associated to one of said scanning positions of the particle beam in the X-Y plane.
a patient support device for positioning said patient in an irradiation treatment position;
an imaging device configured for acquiring an actual position of said moving target while the patient is in said irradiation treatment position, said imaging device comprising a display and displaying controls configured for displaying, in real time, information indicative of the actual position of said moving target and information indicative of a prescribed target position on said display;
breath holding means for holding a breath of said patient while said patient is positioned in said irradiation treatment position;
means for actuating the breath holding means to start holding the patients breath;
means for starting the irradiation of the target with the particle beam.

13. A particle therapy assembly according to claim 12 wherein each of said filtering elements comprises multiple sub-filtering elements having different material thicknesses.

14. A particle therapy assembly according to claim 13 wherein the beam generator and the scanning device are configured for irradiating the target with the particle beam in 20 seconds or less.

15. A particle therapy assembly for irradiating a moving target in a patient with a particle beam, said assembly comprising
a particle beam generator for generating a particle beam having;
a scanning device for scanning said target with said particle beam;
a patient support device for positioning said patient in an irradiation treatment position;
an imaging device configured for acquiring an actual position of said moving target while the patient is in said irradiation treatment position, said imaging device comprising a display and displaying controls configured for displaying, in real time, information indicative of the actual position of said moving target and information indicative of a prescribed target position on said display;
breath holding means for holding a breath of said patient while said patient is positioned in said irradiation treatment position;
means for actuating said breath holding means to start holding the patients breath;
means for starting an irradiation of the target with the particle beam.

16. A particle therapy assembly according to claim 15 wherein said particle therapy assembly further comprises means for positioning said display such that said patient can visually observe said display while said patient is positioned in said irradiation treatment.

17. A method for irradiating a moving target in a patient with a particle beam, said method comprising the steps of
a. positioning said patient in an irradiation treatment position;
b. providing breath holding means for holding the patient's breath;
c. providing an imaging device configured for acquiring an actual position of said moving target while the patient is in said irradiation treatment position, said imaging device comprising a display and displaying controls configured for displaying, in real time, information indicative of the actual position of said moving target and information indicative of a prescribed target position on said display;
d. positioning said display such that the patient can visually observe the display while said patient is located in said irradiation treatment position;
e. displaying, in real time, the information indicative of the actual target position together with the information indicative of the prescribed target position on said display;
f. managing the patient's respiration such that the actual target position matches the prescribed target position within a tolerance, said managing being performed based on said information indicative of the actual target position and said information indicative of said prescribed target position;
g. actuating the breath holding means to start holding the patient's breath when said actual target position matches the prescribed target position within a tolerance;
h. starting the irradiation of the target with said particle beam when said actual target position matches the prescribed target position within said tolerance.

18. A method according to claim 17 wherein
i. the irradiation of the target is completed within a single breath holding period.

19. A method for irradiating a moving target in a patient with a particle beam using a particle therapy assembly comprising
a particle beam generator for generating a particle beam;
a scanning device for scanning the target with the particle beam, said scanning device is comprising
one or more scanning magnets for scanning the particle beam over an X-Y scanning plane;
scanning control means configured for scanning the particle beam by sequentially moving the particle beam to multiple scanning positions situated in said X-Y scanning plane;
said method comprising the steps of
a. positioning said patient in an irradiation treatment position;
b. providing breath holding means for holding the patient's breath;
c. providing an imaging device for acquiring and visualizing an actual target position of said moving target while the patient is in said treatment position and said imaging device comprises a display and displaying controls configured for displaying, in real time, information indicative of the actual target position and information indicative of a prescribed target position;
d. positioning said display such that the patient can visually observe the display while said patient is located in said irradiation treatment position;
e. displaying, in real time, the information indicative of the actual target position together with the information indicative of the prescribed target position on said display;
f. managing the patient's respiratory such that the actual target position matches the prescribed target position within a tolerance, said managing being performed based on said information indicative of the actual target position and said information indicative of said prescribed target position;
g. actuating the breath holding means to start holding the patients breath when said actual target position matches the prescribed target position within a tolerance;
h. starting the irradiation of the target when said actual target position matches the prescribed target position within said tolerance.

20. A method according to claim 19 characterized in that said actuation of the breath holding means is performed by a person other than said patient.

21. A method according to claim 19 characterized in that said actuation of the breath holding means is performed by said patient while being located in said treatment position.

22. A method according to claim 19 wherein the actuation of the breath holding means and the starting of the irradiation are performed simultaneously.

23. A method according to claim 19 wherein said particle therapy assembly further comprises means for modulating an energy of the particle beam as function of said scanning positions and said means for modulating the energy are located downstream of all of said one or more scanning magnets and wherein
i. the irradiation of the target within a single breath holding period.

24. A method according to claim 23 wherein said means for modulating the energy comprises an energy filter, said energy filter having a plurality of filtering elements, each of said plurality of filtering elements being configured for modulating the energy of the particle beam and being associated to one of said scanning positions of the particle beam in the X-Y plane.
